(19) United States
(12) Patent Application Publication
(10) Pub. No.: US 2021/0161775 A1
(43) Pub. Date: Jun. 3, 2021

(54) WATER-RESISTANT COSMETIC COMPOSITION

(71) Applicant: COATEX, Genay (FR)

(72) Inventor: Clémentine CHAMPAGNE, Caluire-et-Cuire (FR)

(73) Assignee: COATEX, Genay (FR)

(21) Appl. No.: 16/979,375

(22) PCT Filed: Mar. 28, 2019
(86) PCT No.: PCT/FR2019/000042

§ 371 Date: Sep. 9, 2020

(30) Foreign Application Priority Data
Mar. 29, 2018 (FR) 18 52731

(51) Int. Cl. A61K 8/06; A61K 8/81; A61Q 1/00; A61Q 5/00; A61Q 17/04; A61Q 19/00

(52) U.S. Cl. CPC A61K 8/062; A61K 8/8147; A61K 8/8152; A61K 8/8194; A61Q 1/00; A61K 2800/805; A61Q 17/04; A61Q 19/00; A61K 2800/10; A61K 2800/33; A61Q 5/00

(57) ABSTRACT

The invention relates to a cosmetic composition in the form of an emulsion which is water-resistant due to the use of an emulsifying polymer. This polymer is prepared by polymerization of an anionic monomer and a C₁-C₇ unsaturated carboxylic acid ester. The cosmetic composition according to the invention is free of film-forming agent and surfactant compound. The invention also relates to the preparation and the use of this cosmetic composition.

The invention relates to a cosmetic composition in the form of an emulsion which is water-resistant due to the use of an emulsifying polymer. This polymer is prepared by polymerisation of an anionic monomer and a C₁-C₇ unsaturated carboxylic acid ester. The cosmetic composition according to the invention is free of any film-forming agents and of surface-active compounds. The invention also relates to the preparation and the use of this cosmetic composition.

There are many cosmetic compositions, particularly cosmetic compositions in the form of oil-in-water emulsions for which a lipophilic phase is dispersed in a hydrophilic phase. These cosmetic compositions in the form of emulsions require the use of a surface-active agent in their preparation.

Such emulsions must be stable and must have, for each use, a specific texture that makes them effective and pleasant to the touch. These cosmetic compositions must also be easy to apply.

Moreover, many known cosmetic compositions comprise film-forming agents that are typically polymers, such as poly(vinylpyrrolidone/eicosene) copolymers, polyurethane acrylate/octylacrylamide copolymers, polyurethanes or acrylate copolymers. These film-forming agents usually provide a water-resistant film after the cosmetic composition has been applied.

However, such film-forming agents usually produce films that feel sticky, greasy or waxy. After application, the film should be smooth and consistent. It must be long-lasting, in particular to avoid overly-frequent applications, but also sufficiently light, in particular to avoid discomfort.

After applying a cosmetic composition, the film applied should be water-resistant, in particular to limit or prevent leaching due to humidity. Water resistance also helps retain the other ingredients in the cosmetic composition more effectively and for a longer time after application, particularly the active or moisturising ingredients in the composition. Moreover, the methods for preparing known cosmetic compositions using surface-active compounds lead to compositions that, after application, produce a film in which the surface-active compound limits the water resistance of these compositions. Indeed, in the presence of humidity, the surface-active compound may produce an emulsion resumption phenomenon that makes the composition soapable. The stability of these cosmetic compositions may thus be impaired due to the presence of a surface-active compound. Document US 2005 0186169 describes a cosmetic method for an anti-wrinkle treatment. This method is based on the use of a film-forming polymer that acts on skin tension. Document WO 2014 139901 relates to sunscreen formulations with enhanced thickening and waterproofing properties. These formulations comprise a styrene polymer. Document CA 2208870 describes a cosmetic or pharmaceutical composition comprising at least one film-forming polymer that provides improved resistance to washing. Document US 2011 0073126 relates to monophasic aqueous cosmetic compositions for transfer-resistant nail polish. They comprise a water-soluble film-forming copolymer and a plasticising copolymer. Document EP 1291001 describes a shampoo composition comprising a cross-linked thickening copolymer of methacrylic acid and C₄-alkyl acrylate and an oil. The article Synthesis, characterisation, and rheological studies of methacrylic acid-ethyl acrylate-diallyl phthalate copolymers (Manjeet Jassal, Badri Narayan Acharya, Pushpa Bajaj, Journal of Applied Polymer Science, May 19, 2003) relates to a study of the synthesis, characterisation and rheology of cross-linked thickening terpolymers. Generally speaking, cosmetic compositions comprise a great many ingredients. The reduction in the number of ingredients while retaining the properties of the cosmetic compositions when used, but also during their preparation, is constantly sought. All of these properties are particularly desirable for cosmetic sunscreen compositions, in particular due to their usual use in wet conditions. This is also true for cosmetic compositions used in makeup, in particular for mascara compositions and foundation compositions.

There is thus a need for improved cosmetic compositions as well as for methods of preparing or using such cosmetic compositions that are also improved.

The cosmetic composition according to the invention provides a solution to all or part of the problems of cosmetic compositions in the prior art.

Thus, the invention provides a cosmetic composition in the form of a water-resistant emulsion that is free of any film-forming agents and of surface-active compounds, and comprising:
particles of a lipophilic phase comprising at least one lipophilic compound, dispersed in:
a continuous hydrophilic phase with a pH greater than or equal to 6.5 and comprising:
   at least one hydrophilic compound and
   at least one emulsifying polymer (P) prepared by at least one polymerisation reaction:
(a1) of at least one anionic monomer comprising at least one polymerisable olefinic unsaturation and at least one carboxylic acid group or one of its salts and
(a2) of at least one C₁-C₇ ester of a compound derived from an acid chosen among acrylic acid, methacrylic acid, maleic acid, maleic anhydride, itaconic acid and crotonic acid.

In the composition according to the invention, the amounts of lipophilic phase and hydrophilic phase may vary, in particular according to the desired texture or according to the final use of the composition.

Preferably, the composition according to the invention comprises from 0.1 to 75% by weight or from 1 to 75% by weight of dispersed lipophilic phase, relative to the total amount by weight of continuous hydrophilic phase and of dispersed lipophilic phase. Also preferably, the composition according to the invention comprises from 0.1 to 70% by weight or from 1 to 70% by weight of dispersed lipophilic phase, relative to the total amount by weight of continuous hydrophilic phase and of dispersed lipophilic phase. More preferably, the composition according to the invention comprises from 0.1 to 65% by weight or from 1 to 65% by weight of dispersed lipophilic phase, relative to the total amount by weight of continuous hydrophilic phase and of dispersed lipophilic phase. Also more preferably, the composition according to the invention comprises from 0.1 to 60% by weight or from 1 to 60% by weight of dispersed lipophilic phase, relative to the total amount by weight of continuous hydrophilic phase and of dispersed lipophilic phase. The cosmetic composition according to the invention comprises at least one lipophilic compound in the form of particles to form the lipophilic phase. Preferably, the lipophilic compound is a cosmetic compound.

Preferably according to the invention, the lipophilic phase comprises at least one lipophilic compound chosen among the fatty acids, fatty alcohols, butters, waxes (for example, beeswaxes), oils, preferably an oil chosen among the mineral oils (for example, paraffin oil, vaseline oil, mineral oils with a boiling point ranging from 300 to 400° C.), animal oils (for example, squalene, squalane, perhydrosqualene), vegetable oils (for example, sweet almond oil, calophyllum oil, palm oil, apricot kernel oil, avocado oil, jojoba oil, olive oil, castor oil, grain germ oils, the liquid fraction of shea butter), unsaponifiable compounds derived from natural oils, synthetic oils (for example, hydrogenated polyisobutene, fatty acid esters such as purcellin oil, butyl myristate, isopropyl myristate, cetyl myristate, isopropyl palmitate, butyl stearate, hexadecyl stearate, isopropyl stearate, octyl stearate, isocetyl stearate, decyl oleate, hexyl laurate, propylene glycol dicaprylate, lanolic acid esters such as disopropyl lanolate, isocetyl lanolate, acetyl glycerides, octanate alcohols, octanate polyalcohols, decanoate alcohols, decanoate polyalcohols in particular octanoate glycols, octanoate glycerols, decanoate glycols, decanoate glycerols, ricinoleate alcohols, ricinoleate polyalcohols), terpenes, polyterpenes, phytosterol, silicone oils (for example, cyclomethicones, low-molecular weight polymethylsiloxanes or silicone oils, high-molecular weight polydimethylsiloxanes or silicone gums, polymethylsiloxanes, dimethiconols, phenyl polydimethylsiloxanes, low-molecular weight siloxanols, high-molecular weight siloxanols, trimethylsiloxysilicates), fluorinated oils (for example, fluorinated perfluoroethers and fluorinated silicones).

In the composition according to the invention, the lipophilic phase is dispersed in the hydrophilic phase which comprises at least one hydrophilic compound. Preferably according to the invention, the hydrophilic compound is chosen among water alone or in combination with at least one compound chosen among glycerol, polyglycerols, glycols, for example, propylene glycol, butylene glycol, moisteners, for example, moisteners for cosmetic compositions, sugar derivatives, for example xylytol, maltilol.

Preferably, the composition according to the invention has a pH greater than 4, more preferentially greater than 5 and much more preferentially greater than 6.

Also preferably the composition according to the invention has a pH less than 13, more preferentially less than 12 and much more preferentially less than 11.

Also preferably, the composition according to the invention has a pH ranging from 4 to 13 or from 4 to 12 or from 4 to 11, more preferentially ranging from 5 to 13 or from 5 to 12 or from 5 to 11, and much more preferentially ranging from 6 to 13 or from 6 to 12 or from 6 to 11.

Essentially according to the invention, the hydrophilic continuous phase comprises at least one hydrophilic compound and at least one emulsifying polymer (P) prepared by at least one polymerisation reaction of monomers (a1) and (a2). Although it has an emulsifying character, the polymer (P) according to the invention is not a surface-active compound.

The polymer (P) used according to the invention is known as such. It may be prepared by known methods, in particular by a radical polymerisation reaction, for example, a polymerisation reaction in an emulsion, a dispersion or a solution. The polymerisation can be carried out in a solvent in the presence of at least one initiator compound. As examples of initiator compounds, at least one compound may be used chosen among the azoic initiators (for example, azobisisobutyronitrile), a peroxide compound, preferably hydrogen peroxide, benzoyl peroxide, benzoyl hydroperoxide, and mixtures thereof Alkaline metal persulphates can also be mentioned, particularly sodium persulphate and potassium persulphate, ammonium persulphate, partially water-soluble peroxides, particularly succinic peracid, t-butyl hydroperoxide, cumyl hydroperoxide, persulphates combined with a copper ion, with a ferrous ion, with a sulphite ion or with a bisulphite ion and mixtures thereof.

Aside from the various monomers, the method for preparing the polymer (P) generally uses at least one chain transfer agent, preferably chosen among the mercaptan compounds, in particular mercaptan compounds comprising at least four carbon atoms such as butyl mercaptan, n-octyl mercaptan, n-dodecyl mercaptan, tent-dodecyl mercaptan, iso-octyl 3-mercaptopropionate. Preferably, the reaction is a radical polymerisation reaction in an emulsion.

According to the invention, the initiator or radical generator compound may therefore be combined with at least one controlled radical polymerisation transfer agent, in particular a Raft-type transfer agent (reversible addition-fragmentation chain transfer or controlled radical polymerisation by reversible chain transfer by addition-fragmentation).

Preferably according to the invention, monomer (a1) is chosen among acrylic acid, methacrylic acid, maleic acid, maleic anhydride, itaconic acid, crotonic acid, an acrylic acid salt, a methacrylic acid salt, a maleic acid salt, a maleic anhydride salt, an itaconic acid salt, a crotonic acid salt and combinations thereof. Much more preferentially, monomer (a1) is chosen among acrylic acid, an acrylic acid salt, methacrylic acid, a methacrylic acid salt and combinations thereof.

Preferably according to the invention, monomer (a2) is a C₁-C₆ ester or a C₁-C₄ ester. Also preferably according to the invention, monomer (a2) is a C₁-C₇ acrylic acid ester or a

C₁-C₇ methacrylic acid ester, more preferentially chosen among methyl acrylate, ethyl acrylate, propyl acrylate, butyl acrylate, ethylhexyl acrylate, methyl methacrylate, ethyl methacrylate, propyl methacrylate, butyl methacrylate, ethylhexyl methacrylate and combinations thereof. More preferentially, monomer (a2) is chosen among ethyl acrylate, butyl acrylate, methyl methacrylate and combinations thereof.

Particularly preferably according to the invention, monomer (a1) is chosen among acrylic acid, methacrylic acid, maleic acid, maleic anhydride, itaconic acid, crotonic acid, an acrylic acid salt, a methacrylic acid salt, a maleic acid salt, a maleic anhydride salt, an itaconic acid salt, a crotonic acid salt and combinations thereof, much more preferentially acrylic acid or methacrylic acid; and monomer (a2) is a C₁-C₆ ester or a C₁-C₄ ester or is a C₁-C₇ acrylic acid ester or a C₁-C₇ methacrylic acid ester, preferably chosen among methyl acrylate, ethyl acrylate, propyl acrylate, butyl acrylate, ethylhexyl acrylate, methyl methacrylate, ethyl methacrylate, propyl methacrylate, butyl methacrylate, ethylhexyl methacrylate and combinations thereof, more preferentially ethyl acrylate, butyl acrylate, methyl methacrylate and combinations thereof.

Also preferably, the polymerisation reaction uses:
from 20 to 70 mol % or from 30 to 60 mol %, preferably from 30 to 55 mol % or from 30 to 50 mol %, of monomer (a1) and
from 30 to 80 mol % or from 40 to 70 mol %, preferably from 45 to 70 mol % or from 50 to 70 mol %, of monomer (a2),
   relative to the total molar amount of monomers (a1) and (a2).

In addition to monomers (a1) and (a2), the polymer (P) can be prepared from other monomers. Thus, the polymer (P) can be prepared by a polymerisation reaction that also uses:
(a3) at least one compound chosen among 2-acrylamido-2-methylpropane sulphonic acid, ethoxym ethacryl ate sulphonic acid, sodium m ethallyl sulphonate, styrene sulphonate hydroxyethyl acrylate phosphate, hydroxypropyl acrylate phosphate, hydroxyethylhexyl acrylate phosphate, hydroxyethyl methacrylate phosphate, hydroxypropyl methacrylate phosphate, hydroxyethylhexyl methacrylate phosphate, their salts and combinations thereof or
(a4) at least one compound chosen among hydroxyethyl acrylate, hydroxypropyl acrylate, hydroxyethylhexyl acrylate, hydroxyethyl methacrylate, hydroxypropyl methacrylate, hydroxyethylhexyl methacrylate or
(a5) at least one cross-linking monomer or at least one monomer comprising at least two olefinic unsaturations.

Also advantageously according to the invention, the polymerisation reaction can use less than 20 mol %, preferably from 0.2 to 20 mol %, particularly from 0.5 to 10 mol %, of monomer (a3) relative to the total molar amount of monomers.

Also advantageously, the polymerisation reaction can use less than 20 mol %, preferably from 0.2 to 20 mol %, particularly from 0.5 to 10 mol %, of monomer (a4) relative to the total molar amount of monomers.

According to the invention, monomer (a5) can be chosen among:
a compound of formula (1):

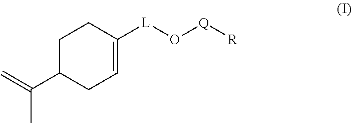

wherein:
   L represents CH₂, CH₂ monoalkoxylate or CH₂ polyalkoxylate, preferably a CH₂ monoethoxylate or CH₂ polyethoxylate group,
   Q represents a direct link or C(O),
   R represents —C(H)═CH₂, —C(CH₃)═CH₂, —C(H)═C(H)C(O)OH, —C(H)═C(H)CH₃, —C(═CH₂)CH₂C(O)OH, —CH₂C(═CH₂)C(O)OH, Q³OQ⁴OC(O)C(CH₃)═CH₂ or Q³OQ⁴OC(O)C(H)═CH₂,
   Q³ represents a difunctional residue of an asymmetric diisocyanate compound, preferably chosen among tolyl-1,3-diisocyanate (TDI) and isophorone-diisocyanate (IPDI) and
   Q⁴ represents CH₂, CH₂—CH₂, CH₂ monoalkoxylate, CH₂—CH₂ monoalkoxylate, CH₂ polyalkoxylate or CH₂-CH₂ polyalkoxylate;
a compound of formula (II):

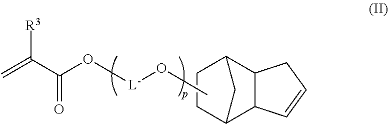

wherein:
   R³ independently represents H or CH₃,
   L¹ independently represents a straight or branched C₁—C₂₀-alkylene group, preferably an ethylene group or a propylene group, and
   p independently represents 0 or an integer ranging from 1 to 30, for example from 1 to 20, particularly from 1 to 15, in particular from 1 to 10.

Monomer (a5) may also be chosen among di(meth)acrylates such as polyalkylene glycol di(meth)acrylate, in particular polypropylene glycol di(meth)acrylate, ethylene glycol di(meth)acrylate, polyethylene glycol di(meth)acrylate, triethylene glycol di (meth)acryl ate, 1,3-butyl ene glycol di (meth)acryl ate, 1,6-butyl ene glycol di(meth)acrylate, 1,6-hexanediol di(meth)acrylate, neopentyl glycol di(meth)acrylate, 1,9-nonanediol di(meth)acrylate, but also 2,2′-bis(4-(acryloxy-propyloxyphenyl))propane, 2,2′-bis(4-(acryloxydiethoxy-phenyl))prop ane and zinc acrylate; tri(meth)acrylate compounds such as trimethylolpropane tri(meth)acrylate and ethoxylated trimethylolpropane tri(meth)acrylate, trimethylolethane tri(meth)acrylate, pentaerythritol tri(meth)acrylate and tetramethylolmethane tri(meth)acrylate; tetra(meth)acrylate compounds such as di-trimethylolpropane tetra(meth)acrylate, tetramethylolmethane tetra(meth)acrylate and pentaerythritol tetra(meth)acrylate; hexa(meth)acrylate compounds such as dipentaerythritol hexa(meth)acrylate; penta(meth)acrylate compounds such as dipentaerythritol penta(meth)acrylate; allyl compounds such as allyl (meth)acrylate, diallyl phthalate, diallyl itaconate, diallyl fumarate, diallyl maleate; polyallyl sucrose ethers with from 2 to 8 groups per molecule, pentaerythritol polyallyl ethers such as pentaerythritol diallyl ether, pentaerythritol triallyl ether and pentaerythritol tetraallyl ether; trimethylolpropane polyallyl ethers such as trimethylolpropane diallyl ether and trimethylolpropane triallyl ether. Other polyunsaturated compounds include divinyl glycol, divinyl benzene, divinylcyclohexyl, and methylenebisacrylamide. Monomer (a5) can also be prepared by an esterification reaction of a polyol with an unsaturated anhydride such as acrylic anhydride, methacrylic anhydride, maleic anhydride, or itaconic anhydride. To obtain monomer (a5), compounds chosen among polyhaloalkanols may also be used such as 1,3-dichloroisopropanol and 1,3-dibromoisopropanol; haloepoxyalkanes such as epichlorohydrin, epibromohydrin, 2-methyl epichlorohydrin and epiiodohydrin; polyglycidyl ethers such as 1,4-butanediol diglycidyl ether, glycerin-1,3-diglycidyl ether, ethylene glycol diglycidyl ether, propylene glycol diglycidyl ether, diethylene glycol diglycidyl ether, neopentyl glycol diglycidyl ether, polypropylene glycol diglycidyl ether, bisphenol A-epichlorohydrin epoxy resin and mixtures thereof.

Monomer (a5) can also be chosen among the trifunctional cross-linking agents. This may be in particular trimethylolpropane tri(meth)acrylate (TMPTA) or trimethylolpropane ethoxylate tri(meth)acrylate (such as TMPTA 3EO).

Monomer (a5) can also be chosen among trimethylolpropane tri(meth)acrylate, ethoxylated trimethylolpropane tri(meth)acrylate, ethylene glycol di(meth)acrylate, methylene(bis)acrylamide, diallyl phthalate, diallyl maleate and mixtures thereof.

Monomer (a5) can also be a mixture of two separate monomers, for example EGDCPEA (ethylene glycol dicyclopentenyl ether acrylate) and TMPTA or EGDCPEA and TMPTA 3EO or even EGDCPEMA (ethylene glycol dicyclopentenyl ether methacrylate) and TMPTA or EGDCPEMA and TMPTA 3EO.

According to the invention, monomer (a5) is preferably chosen among a compound of formula (I), a compound of formula (II), trimethylolpropane trimethacrylate, trimethylolpropane triacrylate, ethoxylated trimethylolpropane trimethacrylate, ethoxylated trimethylolpropane triacrylate, ethylene glycol dimethacrylate, ethylene glycol diacrylate, methylene(bis)acrylamide, diallyl phthalate, diallyl maleate and mixtures thereof.

Also advantageously, the polymerisation reaction can use less than 5 mol %, preferably from 0.01 to 4 mol %, particularly from 0.02 to 4 mol % or from 0.02 to 2 mol %, in particular from 0.02 to 1 mol %, of monomer (a5) relative to the total molar amount of monomers.

Preferably, the polymer (P) is prepared from the sole monomers (a1) to (a5), particularly from combinations of the sole monomers (a1)-(a2), (a1)-(a2)-(a3), (a1)-(a2)-(a4), (a1)-(a2)-(a5), (a1)-(a2)-(a3)-(a4), (a1)-(a2)-(a3)-(a5), (a1)-(a2)-(a4)-(a5), (a1)-(a2)-(a3)-(a4)-(a5). The polymer (P) therefore does not comprise any straight or branched C—C₃₂-alkyl-(EO)ₙ(meth)acrylate monomers comprising ethoxylated n-groups, n ranging from 1 to 150. In particular, it does not comprise any straight (C₁₆—C₁₈)-alkyl-(EO)25methacrylate monomers.

More preferably, the polymer (P) is prepared from the sole monomers (a1) and (a2) or from the sole monomers (a1), (a2) and (a5).

Advantageously according to the invention, the polymerisation reaction uses:
from 19.9 to 66 mol % or from 29.8 to 66 mol %, preferably from 29.8 to 53 mol % or from 29.8 to 49 mol %, of monomer (a1),
from 30 to 80 mol % or from 40 to 70 mol %, preferably from 45 to 70 mol % or from 50 to 70 mol %, of monomer (a2),
from 0.01 to 4 mol % or from 0.02 to 4 mol %, preferably from 0.02 to 2 mol % or from 0.02 to 1 mol %, of monomer (a5),
   relative to the total molar amount of these three monomers.

Preferably according to the invention, the polymer (P) can be fully or partially neutralised, preferably by means of at least one compound chosen among NaOH, KOH, ammonium derivatives, ammonia, amine bases, for example triethanolamine, aminomethyl propanol, or 2-amino-2-methyl-propanol (AMP) and combinations thereof. Also preferably according to the invention, the polymer (P) can be fully or partially coacervated, more preferentially:
by reducing the pH of the dispersion (D), for example by reducing the pH to a value of less than 6.5, in particular by means of an acid compound, in particular by means of least one organic or inorganic acid compound, in particular an acid compound chosen among phosphoric acid, citric acid, glucono-lactone, lactic acid, salicylic acid, glycolic acid, ascorbic acid, glutamic acid, hydrochloric acid, acetic acid, D-gluconic acid, sulphonic acid, methanesulphonic acid, benzimidazole sulphonic acid, tartaric acid, 4-aminobenzoic acid, benzoic acid, sorbic acid, phenylbenzimidazole sulphonic acid, benzylidene camphor sulphonic acid, terephthalylidene dicamphor sulphonic acid or
by increasing the ionic strength of the dispersion (D), for example, by adding at least one ionised compound or at least one salt, particularly NaCl, KCl, MgCl₂, CaCl₂, MgSO₄, CaSO₄.

In the composition according to the invention, the amount of polymer (P) present in the hydrophilic phase may vary. Preferably, the composition according to the invention comprises from 0.4 to 10% by weight, from 0.4 to 9% by weight or from 0.4 to 8% by weight, preferably from 0.5 to 10% by weight, from 0.5 to 9% by weight or from 0.5 to 8% by weight or from 0.6 to 10% by weight, from 0.6 to 9% by weight or from 0.6 to 8% by weight, of polymer (P) relative to the amount of hydrophilic phase.

In addition to the lipophilic and hydrophilic phases, the composition according to the invention may comprise other substances, in particular other substances used in cosmetics. Preferably according to the invention, the composition also comprises at least one substance chosen among mineral particles (for example, titanium dioxide particles, iron oxide particles, zinc oxide particles), coated mineral particles, in particular coated with a hydrophobic compound, for example stearic acid (for example titanium dioxide particles coated with a hydrophobic compound, for example stearic acid), an organic sunscreen (for example chosen among avobenzone, ethylhexyltriazone, benzophenone-3, octocrylene, benzophenone-2, benzophenone-4, ethylhexyl salicyl ate, 4-methylbenzylidene camphor, octyl-N,N-dimethyl PABA (para-aminobenzoic acid), ethylhexyl dimethyl PABA, ethylhexyl methoxycinnamate, isoamyl methoxycinnamate, butyl methoxydibenzoylmethane, diethylamino hydroxybenzoyl hexyl benzoate, bis-ethylhexyloxyphenol methoxyphenyl triazine, drometrizole trisiloxane, methylene bis-benzotriazolyl tetramethylbutylphenol, diethylhexyl butamido triazone, ethylhexyl triazone, isoamyl p-methoxycinnamate, polysilicone-15, tris-biphenyl triazine, homomenthyl salicylate, PEG-25 PABA), active lipophilic cosmetic molecules, active water-soluble cosmetic molecules.

The polymer (P) used according to the invention is not a surface-active compound. A surface-active compound works by forming a monolayer at the water-to-oil interface that reduces the surface tension of the particles of lipophilic compound. This surface-active compound forms micelles. For this, a molecule of surface-active compound comprises two distinct portions: a lipophilic portion and a hydrophilic portion.

Advantageously according to the invention, the polymer (P) makes it possible to control the texture and stability of the emulsion according to the invention. It also enables the composition according to the invention to remain on the surface of the skin after its application, despite the presence of water, for example in case of washing or in the presence of sweat. When applied, the cosmetic composition according to the invention forms a thin layer of lipophilic compound on the surface of the skin. This property can be obtained in the absence of any film-forming agents. The composition according to the invention therefore has good water resistance.

Advantageously, the composition according to the invention can be used in a formulation. The invention therefore also relates to a formulation comprising at least one cosmetic composition according to the invention. Preferably, the formulation according to the invention is chosen among a sunscreen formulation, a makeup formulation, a skin care formulation and a hair care formulation.

In addition to the cosmetic composition according to the invention and this formulation, the invention also relates to the preparation of the cosmetic composition according to the invention. Thus, the invention provides a method of preparing a cosmetic composition in the form of a water-resistant emulsion that is free of any film-forming agents and surface-active compounds and comprising:
the preparation of a continuous hydrophilic phase comprising:
   at least one hydrophilic compound with a pH greater than or equal to 6.5 and
   at least one emulsifying polymer (P) prepared by at least one polymerisation reaction:
      (a1) of at least one anionic monomer comprising at least one polymeri sable olefinic unsaturati on and at least one carboxylic acid group or one of its salts and
      (a2) of at least one C₁—C₇ ester of a compound derived from an acid chosen among acrylic acid, methacrylic acid, maleic acid, maleic anhydride, itaconic acid and crotonic acid,
the preparation of a continuous hydrophilic phase comprising at least one lipophilic compound, then
the addition under stirring and in the absence of a surface-active compound of the lipophilic phase in the hydrophilic phase.

Particularly advantageously according to the invention and due to the emulsifying nature of the polymer (P), the cosmetic composition is prepared in the absence of any surface-active compounds usually used when preparing oil-in-water compositions in the prior art.

Preferably, the addition is carried out at a temperature ranging from 10 to 90° C. or from 15 to 75° C. or at room temperature.

Also preferably, the preparation method according to the invention also comprises the adjustment of the final pH of the composition. Preferably the pH is adjusted to a value ranging from 4 to 13 or from 4 to 12 or from 4 to 11. More preferably, the pH is adjusted to a value ranging from 5 to 13 or from 5 to 12 or from 5 to 11. Much more preferentially, the pH is adjusted to a value ranging from 6 to 13 or from 6 to 12 or from 6 to 11.

When preparing the cosmetic composition according to the invention, the addition of the lipophilic phase in the hydrophilic phase is carried out under stirring, preferably under stirring with a device producing a shear gradient of less than 5,000 s⁻¹.

The particular, advantageous or preferred characteristics of the cosmetic composition according to the invention define preparation methods according to the invention which are also particular, advantageous or preferred.

Furthermore, the invention also relates to a method of improving the water resistance of a cosmetic composition in the form of an emulsion, free of film-forming agents and of surface-active compounds, comprising the addition in the cosmetic composition of at least one emulsifying polymer (P) prepared by at least one polymerisation reaction:
(a1) of at least one anionic monomer comprising at least one polymerisable olefinic unsaturation and at least one carboxylic acid group or one of its salts and
(a2) of at least one C₁—C₇ ester of a compound derived from an acid chosen among acrylic acid, methacrylic acid, maleic acid, maleic anhydride, itaconic acid and crotonic acid.

The particular, advantageous or preferred characteristics of the cosmetic composition according to the invention define methods for improving the water resistance of a cosmetic composition according to the invention which are also particular, advantageous or preferred.

The invention also provides a method of cosmetic treatment using a cosmetic composition according to the invention or by means of a formulation according to the invention or by means of a composition prepared according to the method of preparing a cosmetic composition according to the invention. The cosmetic treatment method according to the invention comprises the application, in particular the application on the skin, of a composition or of this formulation according to the invention.

When used, particularly according to the cosmetic treatment method according to the invention, the cosmetic composition according to the invention makes it possible to obtain an applied layer or an applied film that is water resistant. Such water resistance is particularly advantageous when the cosmetic composition according to the invention is applied to the skin.

The particular, advantageous or preferred characteristics of the cosmetic composition according to the invention define cosmetic treatment methods according to the invention which are also particular, advantageous or preferred.

### EXAMPLES

The following examples illustrate the various aspects of the invention. The following abbreviations are used:
MAA: Methacrylic Acid,
EA: Ethyl Acrylate,
DAP: Diallyl Phthalate,
Clariant Polyglykol B11/50: propylene oxide ethylene oxide monobutyl ether,
Huntsmann Empicol LXVN: sodium lauryl sulphate (SLS),
BASF Texapon NS0: ammonium laureth sulphate in 28% solution or ammonium lauryl ether sulphate in 28% solution (SLES),
sodium persulphate (NH₄)₂S₂O₈,
straight (C₁₆—C₁₈)-alkyl-(EO)25-methacrylate (monomer (x)).

### Example 1

### Preparation of Polymers (P1) according to the Invention and Comparative Polymer (CP1) according to a Semi-Batch Method

In a stirred 1L reactor heated using an oil bath, mixture 1 is prepared by introducing deionised water and a sodium lauryl sulphate (SLS) solution or an aqueous solution containing 28% by mass of sodium lauryl ether sulphate (SLES), and optionally propylene oxide ethylene oxide monobutyl ether (B11/50).

A mixture 2, called a monomer premixture, comprising deionised water, is prepared in a beaker:
monomer (a1), methacrylic acid (MAA),
monomer (a2), ethyl acrylate (EA),
monomer (a5), diallyl phthalate,
optionally monomer (x),
optionally 28% solution of sodium lauryl ether sulphate (SLES) or sodium lauryl sulphate (SLS),
optionally an associative monomer (x), straight (C₁₆—C₁₈)-alkyl-(EO)25-methacrylate.

This premixture is stirred to form a monomer mixture.

An initiator solution is prepared comprising ammonium persulphate and deionised water. All reagents and amounts used are shown in Table 1.

The initiator solution and the monomer premixture are injected in parallel, over two hours, into the reactor heated to 85° C.±1° C. This mixture is baked for 30 min at 85° C.±1° C. and then cooled to room temperature.

The polymers according to the invention and the comparative polymer were prepared under these conditions by varying the monomer compositions of the monomer premixtures. The compositions of the copolymers obtained are shown in Table 1.

TABLE 1
Polymer

| Quantity (g) | | P1 | CP1 |
|---|---|---|---|
| Mixture 1 | deionised water | 400 | 400 |
| | SLS | 2.60 | 0 |
| | SLES | 0 | 6.17 |
| | Polyglykol B11/50 | 1.10 | 0 |
| Premixture | deionised water | 173.70 | 175.00 |
| | SLS | 1.81 | 0 |
| | SLES | 0 | 4.29 |
| | Polyglykol B11/50 | 1.04 | 0 |
| | MAA (a1) | 105.69 | 88.15 |
| | EA (a2) | 191.28 | 176.36 |
| | diallyl phthalate (a5) | 2.75 | 0.88 |
| | monomer (x) | 0 | 22.96 |
| Initiator | deionised water | 54.32 | 62.85 |
| | ammonium persulphate | 0.58 | 0.41 |
| Composition | monomer (a1) (mol %) | 38.98 | 32.51 |
| | monomer (a2) (mol %) | 60.67 | 55.93 |
| | monomer (a5) (mol %) | 0.35 | 0.11 |
| | monomer (x) (mol %) | 0 | 0.52 |
| Characteristics | final solids content (% by weight) | 30 | 29 |
| | particle size (nm) | 74 | 75 |

### Example 2

### Preparation and Evaluation of Cosmetic Compositions (C1 to C8) according to the Invention and Comparative Compositions (CC1 to CC8)

In a beaker, the hydrophilic phase is prepared by mixing, under stirring using an Ika or Rayneri blade stirrer and motor, deionised water and a polymer (P1) according to the invention or a comparative polymer (C1) or comparative polymers of the prior art. The pH is adjusted to 7 by adding a 20% sodium hydroxide solution in water (to pH 5.5 for compositions CC6 and CC8).

The hydrophilic phase of the compositions according to the invention and of the comparative compositions is prepared under stirring at 700-1,000 rpm (except composition CC8 at 1,600-2,000 rpm).

The comparative polymers are polymer (CP1) from example 1 and the comparative polymers of the following prior art:
polymer (CP2): Pemulen TR1 (Lubrizol-INCI: Acrylates/C₁₀₋₃₀ alkyl acrylate crosspolymer),
polymer (CP3): Rheomer SC Plus (Solvay-INCI: acrylates/beheneth-25 methacrylate copolymer).

Comparative composition (CC1) comprises polymer (P1) according to the invention and a surface-active compound (Gattefossé Emulium Delta—INCI: cethyl alcohol (and) glyceryl stearate (and) PEG75 stearate (and) ceteth-20 (and) steareth-20) whereas comparative composition (CC8) does not comprise any polymer and comprises a surface-active compound (Gattefossé Emulium Delta).

The lipophilic compounds of the lipophilic phase are:
Caprylis (INCI: caprylic/capric triglyceride),
commercial hydrogenated sunflower oil (INCI: Helianthus annuus (sunflower) seed oil),
commercial liquid paraffin (INCI: Paraffinum liquidum),
Floramac 10 (Floratech-INCI: Ethyl macadamiate),
Gran sil GMD-3 (Grant Industries-INCI: dimethicone & poly silicone-11).

The ingredients and amounts are shown in Tables 2 and 3 along with the characteristics and properties of the cosmetic compositions prepared. The compositions are stable after one month at 40° C. after being visually assessed by absence of partial separation of the lipophilic phase dispersed on the surface of the hydrophilic phase.

Brookfield viscosity is measured at 20 rpm and 25° C.

To measure the particle size distribution of the dispersed lipophilic phase (D50 in %) in the hydrophilic phase, a Malvern Mastersizer 2000 device is used. D50% is the size for which 50% of the particle volume has a size that is smaller than this particular value.

To evaluate the water resistance of the cosmetic compositions, a Leneta contrast chart is placed on the composition using a 300 μm wedge and allowed to dry for 24 hours. Then, a few drops of deionised water are deposited on the composition film. If the composition film turns whitish, a redispersion phenomenon is taking place, which shows that the cosmetic composition is not stable. On the other hand, the absence of any colour change demonstrates the stability of the cosmetic composition.

TABLE 2

| | Composition according to the invention (g) | | | |
|---|---|---|---|---|
| | C1 | C2 | C3 | C4 |
| hydrophilic phase: | 76 | 74 | 72 | 74 |
| deionised water | | | | |
| hydrophilic phase: | 4 | 6 | 8 | 6 |
| P1 at 30% in water | | | | |
| lipophilic phase: | 20 | 20 | 20 | 60 |
| Caprylis | | | | |
| Viscosity (mPa · s) | 5,570 | 11,900 | 20,400 | 20,800 |
| D50% (μm) | 16.4 | 11.0 | 9.2 | 9.8 |
| water resistance | yes | yes | yes | yes |

| | Composition according to the invention (g) | | | |
|---|---|---|---|---|
| | C5 | C6 | C7 | C8 |
| hydrophilic phase: | 74 | 74 | 74 | 74 |
| deionised water | | | | |
| hydrophilic phase: | 6 | 6 | 6 | 6 |
| P1 at 30% in water | | | | |
| lipophilic phase: | 20 | 0 | 0 | 0 |
| hydrogenated sunflower oil | | | | |
| lipophilic phase: | 0 | 20 | 0 | 0 |
| liquid paraffin | | | | |
| lipophilic phase: | 0 | 0 | 20 | 0 |
| Floramac 10 | | | | |
| lipophilic phase: | 0 | 0 | 0 | 20 |
| Gransil GMD-3 | | | | |
| Viscosity (mPa · s) | 48,090 | 18,660 | 1,950 | 24,300 |
| D50% (μm) | 17.3 | 17.2 | 5.0 | 52.7 |
| water resistance | yes | yes | yes | yes |

TABLE 3

| | Comparative composition (g) | | | |
|---|---|---|---|---|
| | CC1 | CC2 | CC3 | CC4 |
| hydrophilic phase: | 72 | 76 | 74 | 72 |
| deionised water | | | | |
| hydrophilic phase: | 6 | 0 | 0 | 0 |
| P1 at 30% in water | | | | |
| hydrophilic phase: | 0 | 4 | 6 | 8 |
| CP1 at 30% in water | | | | |
| hydrophilic phase: | 2 | 0 | 0 | 0 |
| Emulium Delta | | | | |
| lipophilic phase: | 20 | 20 | 20 | 20 |
| Caprylis | | | | |
| Viscosity (mPa · s) | 32,200 | 6,920 | 29,000 | 47,600 |
| D50% (μm) | 5.7 | 10.9 | 5.0 | 4.3 |
| water resistance | no | no | no | no |

| | Comparative composition (g) | | | |
|---|---|---|---|---|
| | CC5 | CC6 | CC7 | CC8 |
| hydrophilic phase: | 74 | 79.6 | 74 | 74 |
| deionised water | | | | |
| hydrophilic phase: | 6 | 0 | 0 | 0 |
| CP1 at 29% in water | | | | |
| hydrophilic phase: | 0 | 0.4 | 0 | 0 |
| CP2 | | | | |
| hydrophilic phase: | 0 | 0 | 6 | 0 |
| CP3 at 28% in water | | | | |
| hydrophilic phase: | 0 | 0 | 0 | 6 |
| Emulium Delta | | | | |
| lipophilic phase: | 60 | 20 | 20 | 20 |
| Caprylis | | | | |
| Viscosity (mPa · s) | 44,400 | 9,660 | 60,400 | 2,300 |
| D50% (μm) | 7.0 | 32.2 | 6.5 | 17.8 |
| water resistance | no | no | no | no |

Thus, polymer (P1) according to the invention makes it possible to prepare cosmetic compositions that are water-resistant after application.

On the other hand, comparative polymer (CP1) comprising monomer (x) does not make it possible to obtain a cosmetic composition that is water-resistant after application.

Likewise, known comparative polymers (CP2) and (CP3) do not make it possible to obtain a cosmetic composition that is water-resistant after application.

Lastly, the use of a surface-active compound in the comparative cosmetic compositions, with or without polymer (P1) according to the invention, does not make it possible to obtain a cosmetic composition that is water-resistant after application.

### Example 3

### Preparation and Evaluation of a Sunscreen Formulation (F1) according to the Invention

A mixture 1 is prepared comprising:
2,2′,2″,2′″-(ethane-1,2-diyldinitrilo)-tetra-acetic (EDTA) acid (0.2 g),
potassium sorbate (0.1 g) and
water (63.8 g).

A mixture 2 is prepared by adding to mixture 1, polymer (P1) according to the invention (1.2 g) and a commercial rheology modifying agent (Coatex Rheostyl 90 N) (0.7 g). The pH is adjusted by adding, under stirring (700-1,000 rpm), sodium hydroxide 10% by weight in water (1 g).

A mixture 3 is prepared comprising:
a fat-soluble organic UVA filter (BASF Uvinul A Plus Granular) (2 g),
a fat-soluble organic UVB filter (Merck Eusolex 2292 KGaA) (6 g),
a broad-spectrum fat-soluble organic UV filter (BASF Tinosorb S) (3 g) and
emollient (BASF Cetiol B) (4 g).

A mixture 4 is prepared comprising:
a water-soluble organic UVB filter (DSM Parsol HS) (2.5 g) and
deionised water (15 g).

Mixture 3 is added to mixture 2 while under stirring, followed by a preservative (phenoxyethanol) (0.5 g).

The sunscreen formulation (F1) according to the invention is obtained with a pH of 6.7. It has a sun protection factor of 30, evaluated using a simulator (BASF Sunscreen Simulator via www. suns cre ensimul ator. b asf. com).

Its water resistance is 91%.

This water resistance is determined in vitro by measuring the evolution of the sun protection factor according to a spectrophotometric method (Kontron UV spectrophotometer equipped with an integration sphere and monochromator, capable of delivering a UV energy flow of between 290 and 400 nm) by measuring the UV energy flow through the formulation, expressed in energy transmission, and comparing this flow to the initial flow. The source and substrate are also taken into consideration.

The formulation (F1) is spread on a substrate (PMMA Sunplate plates Helioscience) with plastic laboratory syringes or with a micro-pipette with capillary and piston (Gilson Microman 250 μL) to obtain a homogeneous film of about 1.3 mg/cm².

When the formulation has dried, the substrate is placed under running water that is temperature-controlled (29° C.) by a double boiler equipped with an adjustable-flow water circulation pump (Ika). Water is run (3 L/min) over the substrate for 15 minutes.

Then, the substrate is dried and measurements are taken with the spectrophotometer.

The sun protection factor (PF in vitro) is expressed based on the full residual UVB and UVA spectrum that traverses the same layer of formulation as that spread on the skin and corrected according to a spectral sunlight characteristic of the skin's reactivity according to the wavelength.

The average protection factor for the studied preparation was obtained by calculating the arithmetical mean of the protection factors in several trials. Water resistance is the SPF ratio in vitro before and after bathing; it must be greater than or equal to 50% for the formulation to be considered water-resistant.

The resulting 91% clearly demonstrates the very high water resistance of this oil-in-water sunscreen formulation prepared using the emulsifying polymer (P 1) alone.

1. A cosmetic composition in form of a water-resistant emulsion that is free of any film-forming agents and of surface-active compounds and comprising:
particles of a lipophilic phase comprising at list one a lipophilic compound, dispersed in:
a continuous hydrophilic phase with a pH greater than or equal to 6.5 and comprising:
a hydrophilic compound and
an emulsifying polymer (P) prepared by at least one polymerisation reaction:
a monomer (a1) of at least one anionic monomer comprising a polymerisable olefinic unsaturation and a carboxylic acid group or one of its salts, and
a monomer (a2) of at least one C₁—C₇ ester of a compound derived from an acid selected from the group consisting of acrylic acid, methacrylic acid, maleic acid, maleic anhydride, itaconic acid and crotonic acid.

2. The composition according to claim 1, comprising from 0.1 to 75% by weight, of dispersed lipophilic phase relative to a total amount by weight of continuous hydrophilic phase and of dispersed lipophilic phase.

3. The composition according to claim 1, wherein the lipophilic phase comprises a lipophilic compound selected from the group consisting of fatty acids, fatty alcohols, butters, waxes, oils).

4. The composition according to claim 1, wherein the hydrophilic compound is chosen among water alone or in combination with at least one compound selected from the group consisting of glycerol, polyglycerols, glycols moisteners, and sugar derivatives.

5. The composition according to claim 1, with:
a pH greater than 4;
a pH less than 13; or
a pH ranging from 4 to 13.

6. The composition according to claim 1, wherein:
the monomer (a1) is selected from the group consisting of acrylic acid, methacrylic acid, maleic acid, maleic anhydride, itaconic acid, crotonic acid, an acrylic acid salt, a methacrylic acid salt, a maleic acid salt, a maleic anhydride salt, an itaconic acid salt, a crotonic acid salt and combinations thereof or
the monomer (a2) is a C₁—C₆ ester, a C₁-C₇ acrylic acid ester, or a C₁-C₇ methacrylic acid ester or
the monomer (a1) is chosen among acrylic acid, methacrylic acid, maleic acid, maleic anhydride, itaconic acid, crotonic acid, an acrylic acid salt, a methacrylic acid salt, a maleic acid salt, a maleic anhydride salt, an itaconic acid salt, a crotonic acid salt and combinations thereof; and monomer (a2) is a C₁—C₆ ester, a C₁—C₇ acrylic acid ester, or a C₁—C₇ methacrylic acid ester or
the polymerisation reaction uses:
from 20 to 70 mol % of the monomer (a1) and
from 30 to 80 mol % of the monomer (a2),
relative to a total molar amount of the monomers (a1) and (a2).

7. The composition according to claim 1, wherein the hydrophilic phase comprises from 0.4 to 10% by weight of polymer (P) relative to an amount of hydrophilic phase.

8. The composition according to claim 1, wherein the emulsifying polymer (P) is prepared by polymerisation reaction which also uses:
monomer (a3), which is at least one compound chosen among 2-acrylamido-2-methylpropane sulphonic acid, ethoxymethacrylate sulphonic acid, sodium methallyl sulphonate, styrene sulphonate, hydroxyethyl acrylate phosphate, hydroxypropyl acrylate phosphate, hydroxyethylhexyl acrylate phosphate, hydroxyethyl methacrylate phosphate, hydroxypropyl methacrylate phosphate, hydroxyethylhexyl methacrylate phosphate, their salts and combinations thereof;
monomer (a4) which is at least one compound chosen among hydroxyethyl-acrylate, hydroxypropyl-acrylate, hydroxyethylhexyl-acrylate, hydroxyethyl-methacrylate, hydroxypropyl-methacrylate, hydroxyethylhexyl-methacrylate or
monomer (a5) which is at least one cross-linking monomer or at least one monomer comprising at least two olefinic unsaturations.

9. The composition according to claim 1, wherein:
the polymer (P) is fully or partially neutralised or
the polymer (P) is fully or partially coacervated ₄.

10. The composition according to claim 1, further comprising mineral particles, coated mineral particles, an organic sunscreen, active lipophilic cosmetic molecules, or active water-soluble cosmetic molecules.

11. A formulation chosen among a sunscreen formulation, a makeup formulation, a skin care formulation and a hair care formulation, comprising at least one cosmetic composition according to claim 1.

12. A method of preparing a cosmetic composition in form of a water-resistant emulsion that is free of any film-forming agents and of surface-active compounds and comprising:
preparing a continuous hydrophilic phase comprising:
a hydrophilic compound with a pH greater than or equal to 6.5 and
an emulsifying polymer (P) prepared by at least one polymerisation reaction:
a monomer (a1) of at least one anionic monomer comprising a polymerisable olefinic unsaturation and a carboxylic acid group or one of its salts and
a monomer (a2) of at least one C₁-C₇ ester of a compound derived from an acid selected from the group consisting of acrylic acid, methacrylic acid, maleic acid, maleic anhydride, itaconic acid and crotonic acid,
preparing a continuous hydrophilic phase comprising a lipophilic compound, then
adding under stirring and in an absence of a surface-active compound of a lipophilic phase in a hydrophilic phase.

13. The method of preparation according to claim 12:
in which the adding is carried out at a temperature ranging from 10 to 90° C. or
also comprising adjusting a final pH of the composition.

14. (canceled)

15. A method of improving the water resistance of a cosmetic composition in form of an emulsion, free of film-forming agents and of surface-active compounds, comprising adding in a cosmetic composition of at least one emulsifying polymer (P) prepared by at least one polymerisation reaction:
monomer (a1) of at least one anionic monomer comprising a polymerisable olefinic unsaturation and a carboxylic acid group or one of its salts and
monomer (a2) of at least one C_{1-c7} ester of a compound derived from an acid chosen among acrylic acid, methacrylic acid, maleic acid, maleic anhydride, itaconic acid and crotonic acid.

16. A method of cosmetic treatment, the method comprising applying the composition according to claim 1.
